(19) United States
(12) Patent Application Publication
(10) Pub. No.: US 2024/0277897 A1
(43) Pub. Date: Aug. 22, 2024

(54) BIOLOGICAL PROSTHESIS INTENDED FOR THE RECONSTRUCTION OF THE NIPPLE-AREOLA COMPLEX

(71) Applicant: APHA BIOMAT, LA ROCHELLE (FR)

(72) Inventors: Guillaume HOFMANSKI, LA ROCHELLE (FR); Anthony PERES, LA ROCHELLE (FR); Laurent LANTIERI, LA ROCHELLE (FR)

(21) Appl. No.: 18/570,730

(22) PCT Filed: Jun. 17, 2022
(86) PCT No.: PCT/EP2022/066562

§ 371 Date: Dec. 15, 2023

(30) Foreign Application Priority Data
Jun. 17, 2021 (FR) FR2106446

(51) Int. Cl. A61L 27/36; A61F 2/12

(52) U.S. Cl. CPC A61L 27/362; A61F 2/12; A61L 2430/04; A61L 2430/40

(57) ABSTRACT

The invention relates to a biological prosthesis (10) consisting of a nipple-areola complex belonging to the family of pigs, cattle or goats, said nipple-areola complex having been treated for use in humans.

### TECHNICAL FIELD

The invention relates to a novel biological prosthesis produced from an animal nipple-areola complex, of use in particular in breast reconstruction after a mastectomy.

### PRIOR ART

A mastectomy is a surgical procedure that consists in removing the breast, in which a tumor is located, in its entirety, including the areola and the nipple. It is known that approximately one out of eight woman will develop invasive breast cancer in their lifetime. A third of these cancers require a mastectomy. The reconstruction of the nipple-areola complex (NAC) is the final step in the post-mastectomy reconstruction process. Various methods exist for recreating the lost anatomical portion, with a risk mainly associated with a loss of projection with flattening of the nipple. It is therefore necessary to combine surgery with prosthetics.

Currently, the surgical techniques used involve autologous skin grafts for the areola and local flaps for the nipple. A tattoo for the areola can also proposed. Taking an autologous graft necessarily involves losing material from another part of the body, and therefore potential complications with no guarantees regarding the aesthetic outcome. Tattooing does not restore the lost body part. However, the reconstruction of the NAC should allow better integration of the reconstructed breast into the patient's personal psychological plan.

A synthetic prosthetic material made of silicone has recently been put on the market (under the name Fixnip). Complications related to the use of silicone implants in breast implants are widely described in the literature. In addition, recent restrictions (ban on macrotextured implants, ASIA syndrome, etc.) argue in favor of using biological materials in reconstructions of this type so the use of a silicone prosthetic material in NAC surgery does not appear to be consistent. Moreover, subcutaneous implants come with a risk of ulceration with exposure to the implant.

There is therefore a need for a prosthesis suitable for NAC reconstruction that is made of a natural material and is straightforward to produce and use.

The objective of the invention is to address this need by providing a biological prosthesis that overcomes the problems of the prior art.

### SUMMARY OF THE INVENTION

To that end, the present invention proposes using a nipple-areola complex taken from an animal.

Animal skin, in particular pig skin, has already been used for a number of years in the production of biological prostheses in wall and breast reconstruction surgery. Nevertheless, the use of animal dermis to produce a prosthesis in the case of NAC reconstruction would require substantial manipulation and assembly of portions of dermis to obtain the desired shape, which appears difficult to achieve both from a technical point of view and from an economic point of view.

The present invention targets a biological prosthesis produced from a very specific part of the animal, matched to the shape of a human NAC, which avoids these problems.

Specifically, the invention targets a biological prosthesis consisting wholly or partly of a nipple-areola complex belonging to the family of pigs, cattle or goats, said nipple-areola complex having been treated for use in humans, preferentially at least by decellularization and/or deantigenization and/or viral inactivation.

The prosthesis according to the invention therefore comprises a planar or substantially planar section, and a projecting section which is preferably arranged substantially in the center of said planar section.

Advantageously, the shape of such a prosthesis is highly suitable for the reconstruction of the NAC in a woman since it is obtained from the anatomical section (NAC) in the animal, without having to manipulate and assemble different portions of the dermis. It is straightforward to produce, and as a biological prosthesis, it replaces the autologous grafts usually used in surgical procedures of this type.

Other features and advantages will emerge from the detailed description of the invention that follows.

### BRIEF DESCRIPTION OF THE FIGURES

FIG. 1 shows a diagram of the prosthesis according to the invention.

FIG. 2 shows a diagram of the part of the animal taken to produce the prosthesis according to the invention.

FIG. 3 shows a photograph of a prosthesis according to the invention.

### DETAILED DESCRIPTION OF THE INVENTION

### Definitions

What is meant by “decellularization” within the meaning of the invention is the removal of cellular elements. In order for the prosthesis to be suitable for implantation in a recipient, the tissues taken from the donor animal are decellularized so as to reduce their immunogenicity. This method involves the removal of the cells from the donor animal while retaining the biological and mechanical qualities of the extracellular matrix.

What is meant by “deantigenization” within the meaning of the invention is the removal of proteins present in the tissue of the donor animal which might not be recognized by the recipient of the prosthesis, thereby causing the implant to be rejected. Specifically, the donor animal can express enzymes or proteins in the tissue which are not expressed by the recipient. It is therefore important to remove these elements, such as in particular galactose alpha-1,3-galactose (alpha-gal) epitope present in pigs, in order to reduce the immune response after implantation in humans.

What is meant by “projection” or “projecting section” within the meaning of the invention is a bump-shaped protrusion. This projection corresponds to the general shape of a nipple.

What is meant by “prosthesis” within the meaning of the invention is a medical device used in surgery.

What is meant by “biological prosthesis” or “bioprosthesis” within the meaning of the invention is a prosthesis obtained from an animal tissue.

### Medical Device According to the Invention

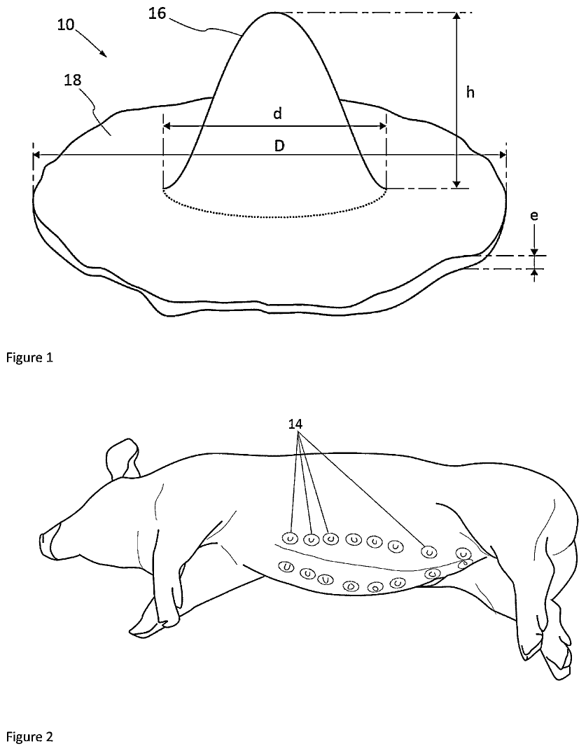

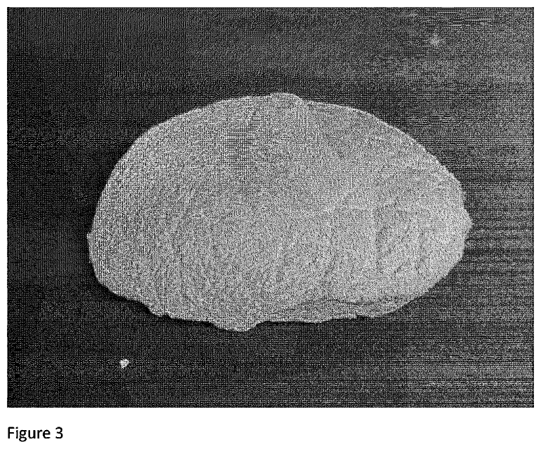

According to a first aspect, the invention therefore targets a biological prosthesis 10 as shown in FIG. 1.

The biological prosthesis according to the invention is a biological prosthesis 10 consisting wholly or partly of a nipple-areola complex 14 of an animal belonging to the family of pigs, cattle or goats, said nipple-areola complex having been treated so as to allow use in humans, preferentially by decellularization and/or deantigenization and/or viral inactivation. The biological prosthesis according to the invention is therefore a biological tissue corresponding to an nipple-areola complex 14 taken from an animal belonging to the family of pigs, cattle or goats, and then treated, preferentially at least by decellularization and/or deantigenization and/or viral inactivation.

The biological prosthesis 10 comprises a planar or substantially planar section 18 (it is not necessarily perfectly planar due to the natural material used), and a projecting section 16 which is preferentially arranged in the center or substantially in the center of said section 18. Section 18 corresponds to the areola and section 16 to the nipple.

Section 18 is preferentially circular or oval, or substantially circular or substantially oval.

The thickness e of section 18 is preferentially between 0.5 and 4 mm, preferably between 0.5 and 2 mm. The thickness e for one and the same prosthesis can vary and a prosthesis can have different thicknesses or be substantially the same thickness, said thicknesses preferentially being between 0.5 and 4 mm, preferably between 0.5 and 2 mm.

This thickness is necessary to be able to ensure good mechanical strength of the implant. The thickness 12 varies with the desired mechanical strength, since the greater the thickness, the less flexible the material. In order to obtain such a thickness, it can be necessary to cut the dermis thicknesswise, to the desired size, using cutting means such as a knife for example.

The projecting section 16 has a height h that is preferentially between 0.5 and 20 mm, preferably between 7 and 10 mm.

The mean diameter D of section 18 is preferentially between 10 and 60 mm, even more preferentially between 15 and 50 mm. Section 18 can have a larger diameter when it is taken and be cut down to size according to the desired final diameter D.

The mean diameter d of section 16 is preferentially between 5 and 20 mm, preferentially between 5 and 15 mm.

These various dimensions of the biological prosthesis are ideal for use in NAC reconstruction surgery. They can be adjusted by the practitioner according to the diameter of the original nipple.

The biological prosthesis according to the invention before treatment is taken from a portion of an animal belonging to the family of pigs, cattle or goats (preferentially a sow) corresponding to the ventral portion of the animal which comprises multiple nipple-areola sections of the animal. Preferentially, it is taken from the ventral portion (abdomen and/or thorax) in one or more regions 14, each region 14 corresponding to a nipple-areola complex.

Animal dermis, and in particular pig dermis, is particularly suitable since its mechanical strength allows its use in surgery. Furthermore, the composition of the extracellular matrix, in particular the collagen, of pig dermis is similar to that of humans.

In order to improve compatibility, the dermis is treated, in particular by decellularization and deantigenization. This treatment allows the removal, in particular, of cellular elements and the alpha-gal epitope which can provoke an elevated immune response and acute rejection of the implant of animal origin.

There are a number of methods known to a person skilled in the art for treating the dermis by decellularization and deantigenization, such as those described in:
Badylak et al., “Extracellular Matrix as a Biological Scaffold Material: Structure and Function,” Acta Biomaterialia (2008), doi:10.1016/j.actbio.2008.09.013
Xu et al., Tissue Engineering, Vol. 15, 1-13 (2009).

The mechanical strength of the biological prosthesis 10 according to the invention preferentially meets the following parameters (measured according to the methods described in the US Pharmacopeia Scaffold Bovine Dermis):
a suture retention strength greater than 5 N,
a tensile strength greater than 5 N/mm2.

In order to be preserved, the biological prosthesis 10 according to the invention can be dried. It is preferably freeze-dried since this drying technique is particularly suited to the preservation of collagen which is a highly temperature-sensitive protein. For use, the prosthesis has to be rehydrated by any method known for this purpose.

The biological prosthesis according to the invention can be produced by means of any suitable method.

Preferentially, it is obtained by means of a production method comprising the following steps:
the integument of the animal is taken from the ventral portion (abdomen and/or thorax) in one or more regions 14, each region 14 corresponding to a nipple-areola.
the dermis is separated from the rest of the integument,
the dermis taken is optionally cut thickness-wise, to obtain the desired thickness e,
the dermis is treated, at least by decellularization and/or deantigenization and/or viral inactivation,
sterilization.

Before being taken, it is necessary to check that the NACs of the animal have diameters D and d greater than or equal to those desired for the prosthesis. This can be measured by any means, for example using a caliper.

The required material is taken from a dead animal, just after it has been slaughtered, directly in the abattoir.

The dermis is separated from the rest of the integument using any cutting means, such as a knife, for example, preferentially directly after being taken in the abattoir.

The dermis is cut using any cutting means, such as a knife, for example, or a slitter. This step must be carried out with a high degree of precision so as to preferentially obtain a substantially uniform thickness across the entire prosthesis.

Before or after sterilization, the method can comprise a step of freeze-drying.

The biological prosthesis according to the invention is preferentially packed in sterile packaging.

Advantageously, the biological prosthesis retains the qualities of the extracellular matrix from which it is extracted and promotes tissue integration, thereby preventing erosion of adjacent tissues. In the event of post-operative and/or recurrent sepsis, the prosthesis according to the invention does not have to be removed unlike synthetic prostheses. Indeed, its biological nature will at worst just result in resorption

The prosthesis is used according to any suitable technique. According to one embodiment, it is used by means of a method comprising the implementation of the following steps:
drawing a circle for positioning the areola according to the position of the contralateral areola
incision over quarter of the circumference
subcutaneous detachment
subcutaneous placement of the implant
closing the approach.

The invention is now illustrated by way of a specific example of taking the material and obtaining the prosthesis, and exemplary biological prostheses according to the invention obtained according to this method.

### Exemplary Method for Producing a Prosthesis According to the Invention

All of the NACs of a sow are taken after slaughter and before any other processing method.

The dermis of the NAC is then separated by knife from the other subcutaneous anatomical elements of the NAC so as to keep as much of the skin as possible.

In order to obtain the desired thickness, the dermis can be cut using a knife or other cutting tools across the entirety of the lower surface and in a uniform manner without cutting into the nipple.

The prosthesis obtained thereby is then treated with various chemical treatments so as to remove cellular elements and other molecules specific to the body of the donor.

The prosthesis is then freeze-dried for optimal preservation but can also be preserved by impregnation with an appropriate chemical solution.

A final cutting operation is carried out in order to obtain the desired final dimensions. The implants are then sterilized by conventional techniques (ETO, radiation, supercritical CO2, peracetic acid).

Exemplary Prostheses Obtained According to this Method

### Example 1

The NAC was taken from a five- to nine-month-old sow and then treated according to the method described above.

The dimensions obtained: h=7 mm; D=50 mm; e=0.9 mm; d=0.5 mm

### Example 2

The NAC was taken from a 20-month-old sow and then treated according to the method described above.

The dimensions obtained: h=25 mm; D=80 mm; e=2 mm; d=15 mm.

1. A biological prosthesis (10) consisting wholly or partly of a nipple-areola complex belonging to the family of pigs, cattle or goats, said nipple-areola complex having been treated for use in humans.

2. The prosthesis (10) of claim 1, wherein it comprises a planar or substantially planar section (18), and a projecting section (16).

3. The biological prosthesis (10) of claim 3, wherein the projecting section (16) is arranged in the center or substantially in the center of section (18).

4. The biological prosthesis (10) of claim 2, wherein section (18) is circular or oval, or substantially circular or substantially oval, in shape.

5. The biological prosthesis (10) of claim 2, wherein the thickness e of section (18) is between 0.5 and 4 mm.

6. The biological prosthesis (10) of claim 2, wherein the projecting section (16) has a height h of between 0.5 and 20 mm.

7. The biological prosthesis (10) of claim 2, wherein the mean diameter D of section (18) is between 10 and 60 mm.

8. The biological prosthesis (10) of claim 2, wherein the mean diameter d of section (16) is between 5 and 15 mm.

9. The biological prosthesis (10) of claim 1, wherein the treatment to allow use in humans is at least one decellularization and/or deantigenization and/or viral inactivation treatment.

10. The biological prosthesis (10) of claim 1, wherein it is freeze-dried.

11. The biological prosthesis of claim 1, wherein it is a breast biological prosthesis for humans.
